SEQUENCE LISTING (5 sequences)

SEQ ID NO: 1 - DNA, 19 bp, Artificial Sequence
  Description of Artificial Sequence Synthetic primer
gctacggcccaacacctta

SEQ ID NO: 2 - DNA, 16 bp, Artificial Sequence
  Description of Artificial Sequence Synthetic primer
ccgacccaccgggata

SEQ ID NO: 3 - DNA, 19 bp, Artificial Sequence
  Description of Artificial Sequence Synthetic primer
ttctggcctggaggctatc

SEQ ID NO: 4 - DNA, 23 bp, Artificial Sequence
  Description of Artificial Sequence Synthetic primer
tcaggaaatttgactttccattc

SEQ ID NO: 5 - DNA, 15 bp, Artificial Sequence
  Description of Artificial Sequence Synthetic oligonucleotide
ttttttttttttttt

(19) United States
(12) Patent Application Publication
(10) Pub. No.: US 2009/0203083 A1
(43) Pub. Date: Aug. 13, 2009

(54) SUBSTRATE FOR NUCLEIC ACID AMPLIFICATION

(76) Inventor: Ralf Mauritz, Penzberg (DE)

Correspondence Address: ROCHE DIAGNOSTICS OPERATIONS INC., 9115 Hague Road, Indianapolis, IN, 46250-0457, US

(21) Appl. No.: 12/200,249

(22) Filed: Aug. 28, 2008

Related U.S. Application Data
(63) Continuation of application No. PCT/EP2007/001659, filed on Feb. 27, 2007.

(30) Foreign Application Priority Data
Mar. 1, 2006 (EP) 06004105.0

(51) Int. Cl. C12P 19/34; C12M 1/00

(52) U.S. Cl. 435/91.2; 435/303.1

(57) ABSTRACT

The present invention relates to a method, a substrate, a kit, and a system for nucleic acid amplification comprising a porous substrate with pores enabling the diffusion of biomolecules. More particular, the present invention relates to a method, a substrate, a kit and a system, wherein the nucleic acid amplification takes place within the pores of a porous substrate.

### RELATED APPLICATIONS

This application is a continuation of PCT/EP2007/001659 filed Feb. 27, 2007 and claims priority to EP 06004105.0 filed Mar. 1, 2006.

### FIELD OF THE INVENTION

The invention relates to a method, a substrate, a kit and a system for nucleic acid amplification comprising a porous substrate with pores enabling the diffusion of biomolecules.

### BACKGROUND OF THE INVENTION

The amplification of nucleic acids is an essential part of almost all diagnostic or analytical tests that are based on nucleic acid analysis. Since the nucleic acids of interest are often present only in very small concentrations, these tests comprise at least one amplification step in order to produce a detectable amount of nucleic acid molecules. A well-known assay which entails the selective binding of two oligonucleotide primers is the polymerase chain reaction (PCR) described in U.S. Pat. No. 4,683,195. This method allows the selective amplification of a specific nucleic acid region to detectable levels by a thermostable polymerase in the presence of deoxynucleotide triphosphates in several cycles.

Since throughput as well as costs are of interest not only for industrial, but also for scientific application, there is a high demand for the parallelization and miniaturization of PCR-based tests. A well-known approach for parallelization of PCR-amplifications is the use of multiwell plates that may be exposed to temperature cycles as a whole by using a thermal block. Here, it is possible to analyze the PCR result directly within the multiwell plates (e.g., by fluorescence) or externally by using, e.g., gel electrophoresis or mass spectrometry. Such multiwell plates allow several 100 reactions in parallel, each with a reaction volume of several μl. Systems for PCR amplifications in multiwell plates with up to 1536 wells are commercially available from several companies.

More recently, special supports with up to 10,000 uniformly distributed holes in a solid support each having a volume of only 50 nl are available (Brenan et al, Proc. SPIE, Vol. 4626, p. 560-569) to perform thousands of different PCR applications in parallel. But of course, the production of such supports with through-holes, the liquid handling, evaporation and cross-contamination between adjacent wells are demanding in such systems.

Applied Biosystems Inc. (Foster City, Calif./USA) introduced a microfluidic card that enables the user to perform PCR reactions for up to eight samples in a plastic disposable with 48 different PCR assays per sample. The primers and hydrolysis probes are presynthesized, spotted into different wells and dried afterwards. For an experiment, the user has to pipette the PCR mastermix including the sample into one well, seal the card with a sealing foil and centrifuge the card such that the PCR mixture can diffuse through a channel system into the different wells before the PCR reactions take place.

Fluidigm (San Francisco, Calif./USA) has developed a system for real-time PCR with a nanofluidic chip for combining 48 samples and 48 assays for a total of 2.304 experiments. After the nanofluidic chip is loaded with samples, sets of primers and FRET probes, the instrumentation automatically combines samples and assays into all possible pairings within discrete 10 nl reaction chambers.

### SUMMARY OF THE INVENTION

In view of the prior art, the invention is directed to a method, a substrate, a kit and a system for nucleic acid amplification, whereby said nucleic acid amplification takes place within the pores of a porous substrate.

One aspect of the present invention is a method for nucleic acid amplification comprising a) providing a porous substrate structured to provide compartments, b) adding a nucleic acid containing sample and an amplification mixture to said porous substrate, and c) exposing said porous substrate to temperature cycles, wherein the nucleic acid amplification takes place within the pores of said porous substrate. Throughout the present invention, nucleic acid amplification summarizes all kinds of amplification procedures known to someone skilled in the art, e.g., the polymerase chain reaction (PCR) described in U.S. Pat. No. 4,683,195. Other possible amplification reactions are the Ligase Chain Reaction (LCR, Wu, D. Y. and Wallace, R. B., Genomics 4 (1989) 560-569 and Barany, Proc. Natl. Acad. Sci. USA 88 (1991) 189-193); Polymerase Ligase Chain Reaction (Barany, PCR Methods and Applic. 1 (1991) 5-16); Gap-LCR (PCT Patent Publication No. WO 90/01069); Repair Chain Reaction (European Patent Publication No. 439 182 A2), 3SR (Kwoh, D. Y. et al., Proc. Natl. Acad. Sci. USA 86 (1989) 1173-1177; Guatelli, J. C. et al., Proc. Natl. Acad. Sci. USA 87 (1990) 1874-1878; PCT Patent Publication No. WO 92/08800), and NASBA (U.S. Pat. No. 5,130,238). Further, there are strand displacement amplification (SDA), transcription mediated amplification (TMA), and Qβ-amplification (for a review see, e.g., Whelen, A. C. and Persing, D. H., Annu. Rev. Microbiol. 50 (1996) 349-373; Abramson; R. D. and Myers, T. W., Current Opinion in Biotechnology 4 (1993) 41-47).

As porous substrate all materials are applicable for the present invention, as long as pores of sufficient dimensions are provided such that the nucleic acid amplification can take place within the pores of said porous substrate. Note that the arrangement of said pores is irrelevant and the substrate can have uniformly or randomly distributed pores as well as pores with uniform or disperse dimension. In other words, the pores are empty spaces within the material of the porous substrate that can be filled with fluids and allow the diffusion of molecules like nucleic acids and enzymes.

In order to enable a nucleic acid amplification within the pores of said porous substrate, it is of course necessary that an exchange of fluids between the substrate and its surrounding is possible and therefore, the material must have pores not only in its interior, but also at its interface. For the nucleic acid amplification to take place within the pores of the porous substrate, said porous substrate must be in physical contact with said nucleic acid containing sample and said amplification mixture. For the subsequent PCR amplification it can be preferred to seal the porous substrate such that the exchange with the surrounding is avoided.

A nucleic acid containing sample summarizes all kinds of nucleic acids in solution throughout the present invention. Said sample may contain one or more types of nucleic acid molecules, optionally together with other biological molecules. The term nucleic acids summarizes DNA, RNA or nucleic acid analogues like locked nucleic acids (LNA) or combinations thereof.

All reagents that are necessary for the nucleic acid amplification reaction are summarized by the phrase amplification mixture throughout the present invention. The amplification mixture may comprise, e.g., enzymes, primers, and nucleotides, together with appropriate buffers, solvents and detergents.

Besides certain requirements with respect to thermal and chemical stability, no other physical parameter restricts the applicability of materials for the present invention. The material can be organic or inorganic, amorphous or crystalline, solid state or plastic as well as elastic or inelastic. Examples are glass fleece, glass fiber, plastics, metal oxides, silicon derivatives, cellulose, nylon, polyester, polypropylene (PP), polyethylene (PE), polyethylenterephthalat (PET), polyacrylnitril (PAT), polyvinylidendifluorid (PVDF) or polystyrene.

The thermal stability of the material is required due to temperature differences that are necessary, e.g., for PCR amplifications. One PCR amplification cycle comprises phases of heating, cooling and phases of constant temperature, whereas the temperature at the beginning of one cycle is the same as the temperature at the end of said cycle. These temperature variations with time are summarized by the phrase temperature cycle, illustrating the cyclic variation of the temperature of said porous substrate. The chemical stability is necessary, because most nucleic acid amplification reactions require certain reagents like buffers or solvents and the porous substrate of the present invention must be resistant with respect to said chemicals.

Another aspect of the present invention is a porous substrate for nucleic acid amplification comprising a) compartments to perform a plurality of individual nucleic acid amplifications in parallel, b) pores enabling the diffusion of nucleic acid molecules and polymerases for a nucleic acid amplification within said pores of the porous substrate and c) at least one primer attached to the surface of said porous substrate.

The surface of the porous substrate summarizes all interfaces of the porous substrate with the surrounding, in other words the outside of the substrate and the inside of the pores. Throughout the present invention, the porous substrate with at least one attached primer can be obtained with certain additional elements, such as means to support the porous substrate in case of a fragile material or means that provide a controlled fluid communication with the porous substrate.

Yet another aspect of the present invention is a multiwell plate for nucleic acid amplification, wherein each well of said multiwell plate comprises a porous substrate according to the present invention such that nucleic acid amplifications take place within said pores of said porous substrates.

A further aspect of the present invention is a kit for nucleic acid amplification comprising a) a porous substrate according to the present invention and b) an amplification mixture.

Still another aspect of the present invention concerns a system for nucleic acid amplification comprising a) a porous substrate according to the present invention and b) a thermocycler.

A thermocycler is an apparatus to expose said device for nucleic acid amplification to temperature cycles. Temperature cycles are necessary for most nucleic acid amplification reactions and therefore, said thermocycler alters the temperature within the porous substrate in such a way that an amplification reaction takes place in the pores of said porous substrate.

Optionally, said thermocycler can have additional means in order to analyze the nucleic acid amplification within the porous substrate.

### DESCRIPTION OF THE FIGURES

FIG. 1: Schematic figure illustrating one embodiment of a porous substrate 1 that is structured by electrochemistry using electrodes 3 in a setup applicable for PCR amplifications comprising compartments 2 with nucleic acids 4 in the pores of the porous substrate and sealings 5, 6 to avoid cross-talk.

FIG. 2: Photographs of two porous substrates with different functionalizations immersed in labeled oligonucleotides.

FIG. 3: Schematic illustration of one embodiment to electrochemically produce a hydrophilic/hydrophobic pattern on a porous substrate 1 using electrodes 3 (x: hydrophobic moiety; U: applied potential).

FIG. 4: Photograph of a functionalized porous substrate immersed in water.

FIG. 5: Fluorescence images of a hybridization cycle

FIG. 6: Fluorescence images of a hybridization cycle

FIG. 7: Gel of PCR products obtained in a standard PCR and a PCR performed within the pores of a porous substrate.

### DETAILED DESCRIPTION OF THE INVENTION

One aspect of the present invention is a method for nucleic acid amplification comprising a) providing a porous substrate structured to provide compartments, b) adding a nucleic acid containing sample and an amplification mixture to said porous substrate, and c) exposing said porous substrate to temperature cycles, wherein the nucleic acid amplification takes place within the pores of said porous substrate.

With respect to the amplification mixture that is necessary for the nucleic acid amplification, the method of the present invention can be performed in at least two different ways. A person skilled in the art knows that enzymes, primers and nucleotides together with appropriate buffers, solvents and/or detergents are needed to perform a nucleic acid amplification.

Therefore, in a preferred method according to the present invention, said amplification mixture comprises enzymes, primers, nucleotides and buffers.

For the nucleic acid amplification to take place within the pores of the porous substrate, said porous substrate must be in physical contact with said nucleic acid containing sample and said amplification mixture. This can be ensured, e.g., by immersing the porous substrate into a solution comprising said nucleic acid containing sample and said amplification mixture or by spotting or pipetting said nucleic acid containing sample and said amplification mixture to defined regions of the porous substrate. The advantage of the spotting or pipetting embodiment is of course that smaller amounts of sample and reagents are needed and that more than one sample can be applied to one porous substrate. It is possible to add said nucleic acid containing sample and said amplification mixture to said porous substrate successively or in one step by several techniques like pipetting, inkjet pin printing or microchannel deposition.

In another preferred method according to the present invention, said porous substrate in step a) is provided with at least one attached primer and/or said amplification mixture comprises enzymes, nucleotides and buffers.

In this embodiment of the present invention, the primers that are necessary for the amplification reaction are already present on the porous substrate prior to adding of the amplification mixture. It is preferred that said primers are attached to the surface of the porous substrate. As mentioned before, the surface of the porous substrate summarizes all interfaces of the porous substrate with the surrounding, in other words the outside of the substrate and the inside of the pores.

An attached primer is a primer that is bound to the surface of the porous substrate. Within the scope of the present invention are all kinds of bonds known to someone skilled in the art. Examples are covalent bonds like, e.g., silane coupling, amide bonds or epoxide coupling, coordinative bindings like, e.g., between His-tags and chelators, bioaffine bindings like, e.g., a biotin/streptavidin bond. Alternatively, the binding of the primers to the porous substrate can be a physisorption. In this embodiment, the primers are applied to the porous substrate simply by, e.g., spotting or pipetting said primers to the substrate followed by evaporation of the solvent.

In a more preferred method according to the present invention, said at least one attached primer is synthesized on the porous substrate.

In another more preferred method according to the present invention, said at least one attached primer is a synthesized primer that is spotted on the porous substrate.

There are mainly two different strategies to provide a porous substrate with at least one attached primer, namely the binding of the entire primer to the substrate (off-chip synthesis) or the synthesis of the primer on the substrate (on-chip synthesis).

For the off-chip synthesis, the porous substrate can be immersed into a solution comprising said primers or by, e.g., spotting or pipetting said primers to defined regions of the porous substrate. If not only a physisorption is required, the subsequent coupling is achieved depending on the used substrate material and the binding moieties of the primers and several alternatives are known to someone skilled in the art. Possible surface modifications can be epoxy functionalizations, like, e.g., epoxysilane derivatives or aldehyde functionalizations or hydroxyl functionalizations or thiole functionalizations or amino functionalizations, like, e.g., amino propyl triethoxy silanes or multi-functional amino coatings (see, e.g., commercial products from Schott Nexterion). To couple spotted primers covalently onto the surface different technologies can be used like photochemical coupling via, e.g., UV mediated cross-linking, wet chemical assisted coupling with appropriate reagents, electrochemistry mediated coupling like, e.g., redox coupling or cross couplings via, e.g., a Diels-Alder reaction.

During the on-chip synthesis the primers are synthesized on the porous substrate in more than one step from single nucleotides, oligonucleotides or polynucleotides (called nucleotide building blocks throughout the invention). Every step of this procedure is called a synthesis cycle throughout this invention.

Preferably, the synthesis or the coupling of primers on the porous substrate is carried out by electrochemical procedures throughout this invention. To realize an electrochemical production of such a porous substrate with attached primers, the porous substrate and/or the nucleotide building blocks have to have binding sites that are protected by protective groups, whereas these protective groups are electrochemically unstable. Therefore, each synthesis cycle of the electrochemical production involves at least one situation, where an electrical potential is applied to the porous substrate, electrochemically deprotecting those protective groups of bindings sites that are electrochemically unstable at the applied potential and that are located at certain parts of the porous substrate and/or at certain nucleotide building blocks already attached to the porous substrate. The deprotection of protective groups can take place by cleaving the entire protective group, cleaving part of the protective group or by a conformational change within the protective group. The electrochemical deprotection of electrochemically unstable protective groups includes the direct deprotection by the applied potential as well as the deprotection by mediators produced at the surface of certain electrodes of the electrode array due to the applied potential. After the deprotection of certain protective groups, single nucleotides, oligonucleotides or polynucleotides can bind to said deprotected binding sites.

In addition, electrodes are necessary to apply electrical potential in order to realize an electrochemical production of such a porous substrate with attached primers. Preferably, said electrodes are arranged in form of an electrode array that comprises a solid support and an arrangement of more than one individual electrode. Any material can be used for these individual electrodes as far as it has an appropriate electrical conductivity and as far as it is electrochemically stable across a certain potential range, namely metallic materials or semiconductor materials. For the solid support of the individual electrodes any material can be used as far as it has properties that avoid a short circuit between individual electrodes.

The arrangement of individual electrodes is designed such that every electrode is a selectively addressable electrode. Therefore, the design of the arrangement of individual electrodes provides the option to address a certain number of electrodes simultaneously in groups or every electrode on its own by an electrical potential.

Every electrode of said electrode array defines a certain area on the porous substrate, where electrochemical reaction can take place due to an applied potential at said electrode. Therefore, every electrode corresponds to an individual spot on the porous substrate, whereas each individual spot comprises certain primers after the electrochemical production resulting in a primer array that can be defined by the production procedure.

Throughout the present invention preferred protective groups are acid labile protective groups, preferably pixyl groups or trityl groups, most preferably 4,4′-dimethoxy triphenylmethyl (DMT) or 4-monomethoxy triphenylmethyl (MMT), or base labile protective groups, preferably levulinyl group or silyl groups, most preferably tert-butyldimethyl silyl (TBDMS) or tert-butyldiphenyl silyl (TBDPS).

In a more preferred method according to the present invention, said attached primers are cleaved from said porous substrate prior to performing said temperature cycles.

In this preferred method according to the present invention, the primers coupled to the porous substrate may be released prior to the nucleic acid amplification. Therefore, the coupling of the primers to the porous substrate must be unstable under certain conditions. The cleavage of the primers from the porous substrate can be performed using electrical potential, irradiation (e.g., UV light), thermal or chemical treatment. Possible cleavable linkers for primers are base-labile moieties like a succinyl-, oxalyl- or a hydrochinone linker (Q-linker), or photo-labile moieties like 2-nitrobenzyl-succinyl- or veratrol-carbonat-linker, or linkers cleavable under reductive conditions like the thio-succinyl-linker, or acid labile moieties like derivatives of trityl groups, for example derivatives of 4,4′-dimethoxy trityl groups.

Note that the nucleic acid amplification can be performed within the pores of said porous substrate with cleaved as well as with attached primers. The nucleic acid amplification takes place during the temperature cycles comprising phases of heating, cooling and phases of constant temperature, whereas the temperature at the beginning of one cycle is the same as the temperature at the end of said cycle. After the last temperature cycle a certain amount of amplified nucleic acids is present within the pores of the porous substrate. Depending on the requirements of the user, there are mainly two different procedures to detect or analyze the amplification product within the porous substrate.

In yet another preferred embodiment of the invention, said amplified nucleic acid is extracted from said porous substrate by centrifugation.

Using the porous substrate according to the present invention, it is possible to remove the amplification product for an external analysis, e.g., with gel electrophoresis, hybridization assays or mass spectrometry.

If an unstructured porous substrate without compartments is used, the entire substrate can be placed in a centrifugation vessel to extract the amplified nucleic acid. If a structured porous substrate with compartments is used, one has to ensure that the amplified nucleic acid from each compartment is collected in separate vessels. This can be achieved by using a microtiter plate that is adjusted to the size and distribution of the compartments of the porous substrate in such a way that each compartment is above a well of a microtiter plate, if the porous substrate is place on top of said microtiter plate.

Alternatively, the porous substrate can be cleaved into several parts, each comprising only one compartment. Afterwards, each of said porous substrate parts can be placed in a separate centrifugation vessel for extracting the respective nucleic acid.

Moreover, the extraction of the amplified nucleic acid can be done by applying a pressure difference (e.g., vacuum) or sucking the liquid off the membrane (e.g., with a pipette).

A preferred embodiment of the invention is a method, wherein the amplified nucleic acid is detected within said porous substrate.

Alternatively, the amplification product can be detected directly in the porous substrate and it is preferred that said detection is based on fluorescence, because the standard techniques to analyze PCR amplifications are based on fluorescence dyes, like intercalating dyes or labeled hybridization probes. In this embodiment, the amplification mixture comprises fluorescent compounds for detecting a respective amplification product. For example, the amplification mixture may comprise several labeled hybridization probes selected from the group consisting of FRET hybridization probes, TaqMan probes, Molecular Beacons and Single labeled probes. Alternatively, a dsDNA binding fluorescent entity such as SYBR Green (Molecular Probes, Inc.), which emits fluorescence only when bound to double stranded nucleic acid may be used.

Moreover, the detection of the amplified nucleic acid within the porous substrate can be performed using electrochemical techniques. In this embodiment, an electrode is placed below the porous substrate to apply an electrical potential and the hybridization probes are labeled with electrochemical moieties, like ferrocen derivatives or osmium complexes.

Furthermore, the detection of the amplified nucleic acid within the porous substrate can be performed using chemiluminescence techniques.

A more preferred embodiment of the invention is a method, wherein the amplified nucleic acid is detected by fluorescence, preferably in real-time.

If the amplification mixture comprises fluorescent probes, it is preferred to monitor the fluorescence of the amplification not only once at the end of the amplification, but at least once in every amplification cycle. In other words, it is preferred to perform a real-time PCR within the pores of the porous substrate.

The method of the invention provides a porous substrate that is structured to provide compartments.

Throughout the present invention compartments are areas of the porous substrate that are separated from each other by impermeable borders. In other words, said impermeable borders surrounding the compartments of the porous substrate avoid the liquid exchange between adjacent compartments. Therefore, it is possible to perform several assays in parallel using only one porous substrate, because the impermeable borders avoid cross-talk between the compartments.

Note that throughout the present invention the structuring of the porous substrate to provide compartments comprises optionally not only the compartments themselves, but also channel structures for the liquid communication between the compartments with the exterior of the membrane. Moreover, it is possible to provide the porous substrate with channel structures that connect two or more compartments, if this is desired for certain applications. The fluid can penetrate the channel structures e.g. by gravitation, capillary forces, pressure or centrifugation.

Another more preferred embodiment of the invention is a method, wherein in each of said compartments individual nucleic acid amplifications are performed.

Using a structured porous substrate, it is of course possible to perform an individual nucleic acid amplification in each of the compartments, whereas there are mainly two different alternatives for this purpose.

In a more preferred method of the invention, said individual nucleic acid amplifications are the same or different.

In another more preferred method of the invention, said different nucleic acid amplifications are based on different samples and/or different primers within said compartments.

One reason to perform the same nucleic acid amplifications in all compartments, may be to generate an increased reliability with respect to the amplification result. Performing a different nucleic acid amplification in each compartment increases the throughput of sample analysis. Different nucleic acid amplifications can be established either by different primers in each compartment that analyzes the same sample or by different samples that are analyzed by a multitude of identical compartments. It is preferred to provide a porous substrate with compartments that each have one or more different primers attached to the surface of the pores within said compartments.

In a preferred embodiment of the method according to the present invention, the porous substrate has an area of 1×10⁻² cm² to 2×10² cm² and a height of 1×10⁻² cm² to 0.5 cm, preferably an area of 1×10⁻¹ cm² to 1×10² cm² and a height of 3×10⁻² cm to 0.3 cm, most preferably an area of 1 cm² to 1×10² cm² and a height of 5×10⁻² cm to 0.2 cm.

With respect to the size and height of the porous substrate several aspects have to be considered. Fist of all, the area of the porous substrate must be large enough to realize the formation of compartments at all and to enable the arrangement of the required number of said compartments. Therefore, the area of the porous substrate is also depending on the intended size of each compartment and the surrounding barriers. The height of the porous substrate must be, on the one hand large enough to provide a certain volume of the compartments in order to perform the PCR amplification and, on the other hand, if a fluorescence technique is used for analysis of the amplification product, thin enough to enable fluorescence detection throughout the entire volume.

In another preferred embodiment of the method according to the present invention, the porous substrate has at least two compartments, preferably between 2 and 1×10⁶ compartments, most preferably between 1×10² and 1×10⁵ compartments.

Another preferred method according to the invention is a method, wherein said compartments are provided by chemical functionalization of said porous substrate.

There are several possibilities to provide a porous substrate with compartments. The phrase chemical functionalization summaries all procedures to chemically modify the surface properties of the porous substrate. It is possible to modify the surface properties of the porous substrate by wet chemical treatments, photochemical treatment, ion bombardment, temperature or by electrochemistry. Note that the chemical functionalization of the porous substrate can be performed directly by the techniques mentioned above or indirectly, where the techniques mentioned above only perform a surface activation such that an additional moiety can bind to said activated binding sites afterwards.

Yet another preferred method according to the invention is a method, wherein said chemical functionalization is performed with electrochemical means.

It is preferred to use electrochemical means, because the surface modification of the porous substrate can be performed in a controlled manner using an electrode array as explained above.

For nucleic acid amplifications in compartments, it is preferred that said compartments are hydrophilic embedded in a hydrophobic surrounding. In general, the procedures to generate a hydrophilic/hydrophobic pattern have to discriminate different areas of the porous substrate.

Technologies that enable the generation of such a pattern are, e.g., electrochemistry by applying a certain current or voltage to certain areas of the porous substrate, photochemistry by applying light of a certain wavelength to certain areas of the porous substrate or spotting technologies by applying a certain volume of reagents to certain areas of the porous substrate.

The starting point for the structuring of the porous substrate can be a preprocessed porous substrate with free functional moieties like, e.g., carboxy, epoxy, aldehyde, hydroxyl amino groups or a preprocessed porous substrate with protected functional moieties like, e.g., the groups mentioned before with respect to the on-chip synthesis of primers that are blocked by a chemical residue or a non-preprocessed porous substrate with no functional groups.

Using a preprocessed porous substrate with free functional groups, electrochemistry, photochemistry or spotting technologies have to address certain areas of the porous substrate in order to attach hydrophilic or hydrophobic moieties.

Using a preprocessed porous substrate with protected functional groups electrochemistry, photochemistry or spotting technologies have to address certain areas of the porous substrate to enable a chemical reaction in order to attach or deprotect a hydrophilic or hydrophobic moiety. For example, a porous substrate with functional moieties protected by hydrophobic groups can be deprotected in order to create hydrophilic areas and the untreated areas will remain hydrophobic. The opposite process with functional moieties protected by hydrophilic groups can be used to generate a pattern by cleaving the hydrophilic protecting groups. To create hydrophobic areas it can be useful to couple additional hydrophobic residues to the deprotected areas after said deprotection.

Using a non-preprocessed porous substrate with no functional groups the hydrophilic/hydrophobic pattern can be generated by modification of certain areas of the porous substrate with functional groups.

To generate a hydrophilic/hydrophobic pattern different groups can be used. For the hydrophilic areas hydrophilic groups like e.g. hydroxyl, amino, carboxy, thiole, phosphate are applicable. For hydrophobic areas hydrophobic groups like e.g. cholesterol, carbon alcohols (e.g. dodecanol), trityl derivatives or palmitoyl are suitable. To enhance the hydrophilic/hydrophobic properties multi-functional residues like dendrimers or branching derivatives can also be used. It is preferred that the hydrophilic/hydrophobic residues are coupled to the porous substrate in a covalently manner in order to provide sufficient stability for the subsequent amplification reaction.

Further preferred is a method according to the invention, wherein said compartments are provided by spotting of fluids.

Fluids that are suitable to structure the porous substrate to provide compartments are materials in solvents that evaporate at atmospheric pressure and thereby form a film of said material. An example for this strategy is a solution of polyvinylchloride (PVC) in tetrahydrofurane (THF).

Note that it is possible to provide a porous substrate with more than one kind of primer molecule per compartment. This can be done by using compartments with orthogonal protective groups for the production of the primer array, whereas said orthogonal protective groups are at least two different protective groups that are unstable under different conditions, e.g. different electrical potentials, acid/base instability or any other combination of electrochemistry, wet chemistry and photochemistry. Using such orthogonal protective groups e.g. for the protection of the binding sites of the porous substrate provides the opportunity to produce a mixture of more than one type of primer in one individual compartment of the porous substrate. The at least two different protective groups can be provided each as an individual surface modification or as a single branched surface modification comprising two or more of said different protective groups.

A preferred method according to the present invention is a method, wherein an additional pre-hybridization step is performed prior to exposing said porous substrate to temperature cycles and prior to the optional cleaving of the primers from said porous substrate.

Providing a primer array has the additional advantage that a pre-hybridization step can be performed prior to the amplification reaction in order to accumulate certain nucleic acids of the applied sample at the corresponding compartments of the structured porous substrate. This pre-hybridization step is a hybridization step that occurs, if the nucleic acids within the sample are in contact with the covalently bound primers of the respective compartments. In other words, each target nucleic acid within the sample finds an attached primer with the complementary sequence prior to the subsequent amplification reaction. Due to such a pre-hybridization step it is possible to detect much lower concentrations, because the detection limit is no longer dependent on the statistical distribution of each nucleic acid across all compartments of the array.

In a more preferred method according to the present invention, said porous substrate is sealed in order to avoid cross-talk between the compartments.

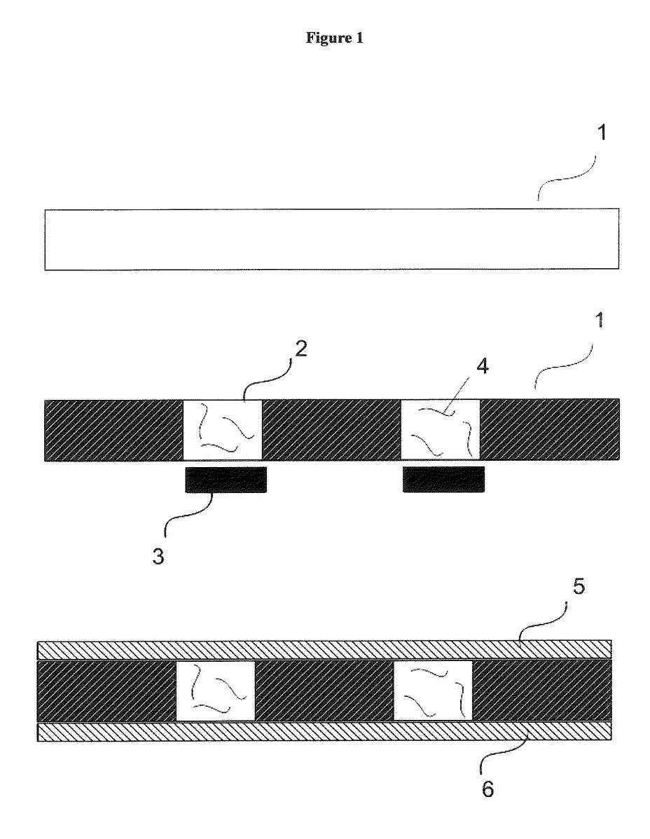

FIG. 1 shows a schematic figure illustrating one embodiment of a porous substrate that is sealed in order to avoid cross-talk between the compartments. If the porous substrate 1 is structured to provide compartments 2, it is of importance to avoid cross-talk between the compartments not only within the porous substrate but also via its surrounding. Therefore, it is preferred to seal the porous substrate after the sample and/or the amplification mixture is applied and prior to the PCR amplification. Throughout the present invention all kinds of sealings 5,6 for aqueous solutions are possible that are known to someone skilled in the art. Examples are e.g. a plastic foil that can be glued to the surface of the porous substrate or glass slides that can be pressed by mechanical force to the porous substrate to provide a water-tight contact. If glass slides are used for sealing the porous substrate, it is preferred to use hydrophobic glass slides (e.g. silanized glass) or an intermediate oil film. Alternatively, the entire porous substrate can be immersed in oil, e.g. PCR oil. Moreover, evaporating fluids that thereby form a film on surfaces, like e.g. polyvinylchloride (PVC) in tetrahydrofurane (THF), are suitable to seal the porous substrate. Note that an optical transparent material has to be used, if e.g. a fluorescence detection of the PCR within the porous substrate is required and that a reversible sealing is necessary, if a subsequent extraction of the amplified nucleic acid is intended.

In a preferred method according to the present invention, a thermal base 6 is used to seal one side of the porous substrate. A thermal base is a special heat pipe in a plate-like form that is commercially available from Thermacore (Lancester, USA) as Therma-Base™. A heat pipe is a sealed vacuum vessel with an inner wick structure that transfers heat by the evaporation and condensation of an internal working fluid. Ammonia, water, acetone, or methanol are typically used, although special fluids are used for cryogenic and high-temperature applications. As heat is absorbed at one side of the heat pipe, the working fluid is vaporized, creating a pressure gradient within the heat pipe. The vapor is forced to flow to the cooler end of the pipe, where it condenses, giving up its latent heat to the wick structure and than to the ambient environment via, e.g., a heat sink. The condensed working fluid returns to the evaporator via gravity or capillary action within the inner wick structure. Because heat pipes exploit the latent heat effect of the working fluid, they can be designed to keep a component near ambient conditions. Though they are most effective when the condensed fluid is working with gravity, heat pipes can work in any orientation.

Therefore, using a thermal base for sealing one side of the porous substrate has additional positive effects with respect to the thermocycling that is necessary for the PCR amplifications within the porous substrate. Some more details with respect to embodiments of the present invention including a thermal base can be found later in the description.

Note that the sequence of sealing the porous substrate after the sample and the amplification mixture are applied can be altered, if the porous substrate is structured to provide compartments and channel structures. In this case, the porous substrate can be sealed, e.g., already after primers are attached to the pores within the compartments. Afterwards, the amplification mixture and the sample are applied to the compartments via the channel structure to perform the amplification reaction.

An additional procedure to provide compartments within the porous substrate is the use of mechanical pressure to partially compress the substrate such that the pores are closed or minimized and the diffusion of liquids is hindered.

Moreover, the porous substrate can be structured to provide compartments by using a temperature or laser treatment that partially melts the porous substrates such that the pores of the porous substrates become closed in the treated area.

In still another preferred method according to the present invention, said porous substrate is a glass fleece, an organic polymer like cellulose or an inorganic polymer like nylon, polyester, polypropylene (PP), polyethylene (PE), poly-ethylenterephthalat (PET), polyacrylnitril (PAT), polyvinylidendifluorid (PVDF) or polystyrene.

Moreover, other materials like glass, metal oxides or silicon derivatives are suitable for the present invention as far as they are processed in such a manner that they provide pores that enable the nucleic acid amplification therein.

Another aspect of the present invention is a porous substrate for nucleic acid amplification comprising a) compartments to perform a plurality of individual nucleic acid amplifications in parallel, b) pores enabling the diffusion of nucleic acid molecules and polymerases for a nucleic acid amplification within said pores of the porous substrate, and c) at least one primer attached to the surface of said porous substrate.

The primers can be attached to the porous substrate by any procedure that is known to someone skilled in the art. Examples are covalent bonds like, e.g., silane coupling, amide bonds, aldehyde or epoxide coupling, cross couplings via, e.g., a Diels-Alder reaction, coordinative bindings like, e.g., between His-tags and chelators, bioaffine bindings like, e.g., a biotin/streptavidin bond. Alternatively, the binding of the primers to the porous substrate can be a physisorption. In this embodiment, the primers are applied to the porous substrate simply by, e.g., spotting or pipetting said primers to the substrate followed by evaporation of the solvent.

In a preferred porous substrate according to the present invention, said primers are attached to the porous substrate covalently.

The porous substrate according to the present invention has compartments to perform a plurality of individual nucleic acid amplifications in parallel.

The different possibilities and requirements for a porous substrate having compartments with or without channel structures have been explained before. Note that if different nucleic acid amplifications are performed in the compartments of the porous substrate, it is preferred to seal the porous substrate after loading of sample, primers and/or probes and prior to the amplification reaction. If channel structures are provided, the porous substrate can alternatively be sealed prior to the loading of sample and amplification mixture.

In yet another preferred porous substrate according to the present invention, said compartments are defined by chemical barriers, preferably said chemical barriers are chemical functionalizations of said porous substrate.

As mentioned before, one possibility to structure the porous substrate is the chemical functionalization of the material of the porous substrate. For example, a certain part of a hydrophilic porous substrate may be altered such that it is hydrophobic afterwards. In other words, the functionalized, hydrophobic part of the hydrophilic porous substrate forms a chemical barrier for aqueous solutions.

In a more preferred porous substrate according to the present invention, said chemical functionalizations are electrochemical functionalizations.

In yet another preferred porous substrate according to the present invention, said compartments are defined by spotting of fluids.

The alternatives of the present invention with respect to electrochemical functionalizations and spotting of fluids to structure the porous substrate in order to provide compartments with or without channel structures were already outlined before.

Another preferred porous substrate according to the present invention is a substrate, wherein each compartment has the same or different attached primers.

In general, the compartmentation of the porous substrate is provided to perform multiple different amplification reactions in parallel and there are mainly two different alternatives, namely the same set of primers and different samples or a different set of primers and the same sample. Therefore, the porous substrate can be provided either with the same set of primers in each compartment in order to screen a plurality of samples or with different primers in each compartment in order to screen a sample for several ingredients.

Another aspect of the present invention is a multiwell plate for nucleic acid amplification, wherein each well of said multiwell plate comprises a porous substrate according to the present invention such that nucleic acid amplifications take place within said pores of said porous substrates.

Using multiwell plates for handling a plurality of porous substrates has the advantage that this setup is applicable for many commercial devices, like blockcycler to perform the amplification reaction in a controlled and highly parallel manner. Additionally multiwell plates are compatible to technologies to increase throughput for screening purposes like, e.g., automatic pipetting technologies using robotic instruments, analyzing technologies with standard detection instruments.

Yet another aspect of the present invention is a kit for nucleic acid amplification comprising a) a porous substrate according to the present invention and b) an amplification mixture.

Throughout the present invention, the amplification mixture comprises all compounds that are necessary to perform a nucleic acid amplification reaction in the form of a Polymerase Chain Reaction (PCR), namely a thermostable DNA polymerase, at least one nucleic acid compound, deoxynucleotides, a buffer with at least one sort of a divalent cation, preferably Mg²⁺. In addition, the amplification mixture may comprise, e.g., a synthetic peptide with a divalent cation binding site for “hot start” PCR or other PCR additives.

In a preferred kit according to the present invention, said amplification mixture comprises enzymes, primers, nucleotides and buffer.

As thermostable polymerases, a great variety of enzymes may be used. Preferably, said thermostable DNA polymerase is selected from a group consisting of Aeropyrum permix, Archaeoglobus fulgidus, Desulfurococcus sp. Tok., Methanobacterium thermoautotrophicum, Methanococcus sp. (e.g., jannaschii, voltae), Methanothermus fervidus, Pyrococcus species (furiosus, species GB-D, woesii, abysii, horikoshii, KOD, Deep Vent, Proofstart), Pyrodictium abyssii, Pyrodictium occultum, Sulfolobus sp. (e.g., acidocaldarius, solfataricus), Thermococcus species (zilligii, barossii, fumicolans, gorgonarius, JDF-3, kodakaraensis KODI, litoralis, species 9 degrees North-7, species JDF-3, gorgonarius, TY), Thermoplasma acidophilum, Thermosipho africanus, Thermotoga sp. (e.g., maritima, neapolitana), Methanobacterium thermoautotrophicum, Thermus species (e.g., aquaticus, brockianus, filiformis, flavus, lacteus, rubens, ruber, thermophilus, ZO5 or Dynazyme). Also within the scope of the present invention are mutants, variants or derivatives thereof, chimeric or “fusion-polymerases”, e.g., Phusion (Finnzymes or New England Biolabs, Catalog No. F-530S) or iProof (Biorad, Cat. No. 172-5300), Pfx Ultima (Invitrogen, Cat. No. 12355012) or Herculase II Fusion (Stratagene, Cat. No. 600675). Furthermore, compositions according to the present invention may comprise blends of one or more of the polymerases mentioned above.

In one embodiment, the thermostable DNA Polymerase is a DNA dependent polymerase. In another embodiment, the thermostable DNA polymerase has additional reverse transcriptase activity and may be used for RT-PCR. One example for such enzyme is Thermos thermophilus (Roche Diagnostics cat. No: 11 480 014 001). Also within the scope of the present invention are blends of one or more of the polymerases compiled above with retroviral reverse transcriptases, e.g., polymerases from MMLV, AMV, AMLV, HIV, EIAV, RSV, and mutants of these reverse transcriptases.

The concentrations of the DNA polymerase, the deoxynucleotide and the other buffer components are present in standard amounts, the concentrations of which are well known in the art. The Mg² concentration may vary between 0.1 mM and 3 mM and is preferably adapted and experimentally optimized. However, since the concentration optimum usually depends on the actual primer sequences used, it can not be predicted theoretically.

The at least one nucleic acid compound of the amplification mixture comprise at least one pair of amplification primers to perform a nucleic acid amplification reaction.

Furthermore, the amplification mixture may comprise fluorescent compounds for detecting a respective amplification product in real time and respectively 2-6 differently labeled hybridization probes not limited to but being selected from a group consisting of FRET hybridization probes, TaqMan probes, Molecular Beacons and Single labeled probes. Alternatively, such an amplification mixture may contain a dsDNA binding fluorescent entity such as SYBR Green, which emits fluorescence only when bound to double stranded DNA.

Moreover, the amplification mixture may be adapted to perform one-step RT-PCR and comprises a blend of Taq DNA Polymerase and a reverse transcriptase such as AMV reverse transcriptase. In a further exemplary particular embodiment, such an amplification mixture is specifically adapted to perform one-step real time RT-PCR and comprises a nucleic acid detecting entity such as SYBR Green or a fluorescently labeled nucleic acid detection probe.

In another preferred kit according to the present invention at least one primer is attached to the surface of said porous substrate and said amplification mixture comprises enzymes and nucleotides.

In this embodiment of the kit, the primers are already attached to the surface of said porous substrate and therefore, the amplification mixture must not contain the primer molecules.

Still another aspect of the present invention is a system for nucleic acid amplification comprising a) a porous substrate according to the present invention and b) a thermocycler.

Throughout the present invention a thermocycler summarizes all components that are necessary to perform thermocycles with the porous substrate. A thermocycler comprises at least one heat pump, e.g., Peltier elements to increase the temperature of the porous substrate, a heat sink to dissipate heat during cooling of the porous substrate and a control unit to control said simultaneous thermocycling of multiple samples. As mentioned before, it is preferred to provide an additional thermal base between the porous substrate and the heat sink in order to increase the velocity and precision of temperature changes as well as to provide a homogeneous heating/cooling procedure across the entire cross-section area of the porous substrate.

In a preferred system according to the present invention, said thermocycler comprises at least one heat pump, a heat sink and/or a control unit.

In another preferred system according to the present invention, said thermocycler comprises an illumination means and a detection means.

It is preferred to provide a system with an additional detection means to analyze the amplification result directly at the porous substrate. It is preferred that said detection means is a fluorescence detector, because the standard techniques to analyze PCR amplifications are based on fluorescence dyes, like intercalating dyes or labeled hybridization probes. If the amplification results should be analyzed with fluorescence techniques, the amplification mixture of the present invention further comprises the fluorescence probe. Since fluorescence techniques do require light for the excitation of the fluorescence dyes, a preferred system according to the present invention further comprises an illumination means.

Depending on the size of the cross-section area of the porous substrate, the fluorescence detector and the illumination means have to fulfill special requirements. If the porous substrate of the present invention has compartments distributed on its cross-section area, one has to assure that compartments in the center of the porous substrate and compartments at its boarder obtain the same illumination and that the fluorescence intensity is recorded in a comparable fashion. This can be achieved by using a fluorescence detector and/or an illumination means equipped with a telecentric optic.

Within the scope of this invention a telecentric optic is an optic having a very small aperture and thus provides a high depth of focus. In other words, the telecentric light of a telecentric optic is quasi-parallel with the chief rays for all points across the object being parallel to the optical axis in object and/or image space. Therefore, the quality of an illumination means or a detection means utilizing telecentricity in the object space is insensible to the distance of a certain object point to the optic. The aperture of a telecentric optic is imaged at infinity. In addition, using telecentric light a good lateral homogeneity across the light beam is assured and the sites located in the center of the assembly are comparable to those located at the boarder of the assembly. Throughout the present invention, a telecentric optic always comprises a field lens. In the context of this invention a field lens is a single lens that is closest to the objective that determines the field of view of the instrument, that comprise one or more components (achromat) and that contributes to the telecentricity in object and/or image space in combination with additional optical components of the apparatus.

The field lens of the present invention transfers excitation light from a light source to the porous substrate and transfers fluorescence signals from the porous substrate to the detector. This does not exclude that additional optical components are introduced in the beam path, e.g., between the light source and the field lens, between the field lens and the detector or between the field lens and the porous substrate.

In another preferred system according to the present invention, said nucleic acid amplification is a real-time PCR.

If the system according to the present invention is equipped with a fluorescence detector and an illumination means, it is preferred to monitor the fluorescence of the amplification not only once at the end of the amplification, but at least once in every amplification cycle. In other words, it is preferred to perform a real-time PCR within the pores of the porous substrate.

Yet another preferred system according to the present invention further comprises a means to extract the amplified nucleic acid from said device for nucleic acid amplification.

In another embodiment of this system according to the present invention, the system is equipped with an additional means to extract the amplified nucleic acid from the porous substrate. In certain embodiments it can be desired to extract the amplified nucleic acid from the porous substrate for a subsequent analysis. Such an external analysis is, e.g., a mass spectrometric analysis or a gel analysis.

In a more preferred system according to the present invention, said means to extract the amplified nucleic acid is a centrifugation means.

The different procedures to extract the amplified nucleic acid from a porous substrate with and without compartments was already explained in detail before.

The following examples, sequence listing and figures are provided to aid the understanding of the present invention, the true scope of which is set forth in the appended claims. It is understood that modifications can be made in the procedures set forth without departing from the spirit of the invention.

### Example 1

### Generation of a Hydrophilic/Hydrophobic Pattern on a Porous Substrate Using an Electrochemical Procedure and Dispensing a Labeled Oligonucleotide to the Substrate

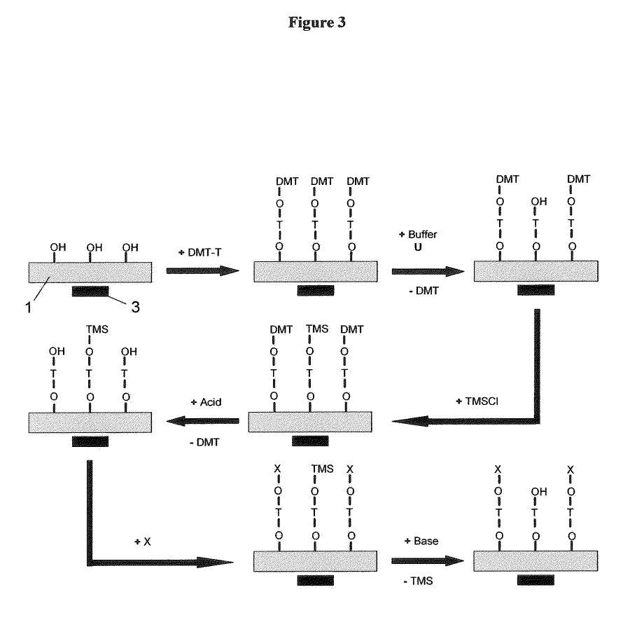

The preparation of a hydrophilic/hydrophobic pattern comprising two hydrophilic positions on a hydrophobic substrate were generated using the electrochemical procedure depicted in FIG. 3. In this example the coupling of a hydrophobic moiety to a hydrophilic functionalized porous substrate is described. For this experiment, a self-made reaction chamber comprising an electrode array with two gold electrodes, an inorganic porous substrate, standard DNA synthesis reagents, phosphoramidites of the hydrophobic moiety and a buffer solution to electrochemically generate an acid media at the activated electrode was used.

The porous substrate (PE-Sinter membrane from PolyAn, Berlin/Germany, pore size: 10 μm, thickness: 0.6 mm; Loading density of hydroxyl groups: 1.7 μmol/cm²) is placed in proximity to the electrodes in the reaction chamber. Because the porous substrate itself has only binding sites without any protective groups, 5′-DMT-T-3′-phosphoramidites were coupled to the porous substrate as a starting group. For this purpose, the 5′-DMT-T-3′-phosphoramidites together with an activator (Dicyanoimidazol in acetonitrile) were filled into the chamber to react with the functional groups of the membrane.

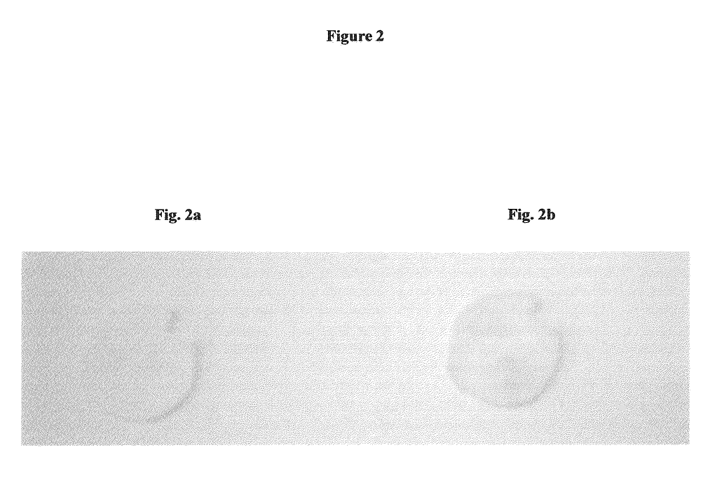

The solution was removed afterwards and an oxidation step was performed in order to oxidize the trivalent phosphor molecule from the first coupling step to the more stable pentavalent phosphor molecule. Then, the oxidation solution was rinsed out of the reaction chamber and a capping step was performed to block all unreacted hydroxyl groups of the porous substrate from the first coupling step for further reactions. Afterwards, the capping solution was removed and the buffer solution was filled into the chamber. To modify the porous substrate with hydrophilic spots and a hydrophobic surrounding (FIG. 2a) the following procedure was used (illustrated in FIG. 3). An electrical potential (−300 μA for 60 sec) was applied to both of the two electrodes successively in order to cleave the protecting groups on those parts of the porous substrate being in proximity to the activated electrodes. Afterwards, the buffer solution was rinsed out of the chamber again and a solution of trimethyl chlorosilane in pyridine was added for 10 minutes to react with the prior deprotected hydroxyl groups. Hence, the hydroxyl groups are blocked by a silyl group which leads to a point where the positions above the electrodes have silyl protected hydroxyl groups and the positions beside the electrodes have trityl protected hydroxyl groups. Thus, an acidic solution of 3% trichloro acetic acid in dichloromethane (DMT-Removal reagent, Roth, Karlsruhe/Germany, Cat. No. 2257,1) was added for two minutes to remove all remaining DMT groups beside the electrodes. Therefore, the deblocked hydroxyl groups were now accessible to react with a hydrophobic moiety to give a hydrophobic area onto the membrane. Thus, a Cholesterol-phosphoramidite (0.1 M solution in acetonitrile of Tetra Ethylene Glycol Cholesterol phosphoramidite, ChemGenes Corp., Wilmington, Mass./USA, Cat. No. CLP-2704) with an activator was filled into the chamber to react at the deprotected binding site of the porous substrate. After two minutes of incubation the phosphoramidite solution was rinsed out and another oxidation step was performed to stabilize the trivalent phosphor moiety. After the exchange of the oxidation solution an aqueous solution of ammonia was flushed into the reaction chamber with an incubation time of 60 minutes to release the silyl protecting groups from the positions above the electrodes and to deprotect all phosphate protecting groups from phosphate moieties. After some subsequent washing steps with acetonitrile and water, a hydrophilic/hydrophobic pattern with hydrophilic properties above the electrodes and hydrophobic properties beside the electrodes was obtained.

To modify the porous substrate with hydrophobic spots and a hydrophilic surrounding (FIG. 2b) a second substrate was prepared under similar conditions, but with the opposite pattern by using the following procedure. After generating the starting layer with the first coupling of 5′-DMT-T-3′-phosphoramidites onto the substrate the buffer solution was filled into the chamber and an electrical potential (−300 μA for 60 sec) was applied to both of the two electrodes successively in order to cleave the protecting groups on those parts of the porous substrate being in proximity to the activated electrodes. Then, a Cholesterol-phosphoramidite (0.1 M solution in acetonitrile of Tetra Ethylene Glycol Cholesterol phosphoramidite, ChemGenes Corp., Wilmington, Mass./USA, Cat. No. CLP-2704) with an activator was filled into the chamber to react at the deprotected binding site of the porous substrate. After two minutes of incubation the phosphoramidite solution was rinsed out and another oxidation step was performed to stabilize the trivalent phosphor moiety. The solution is removed afterwards and a capping reaction was performed to block all unreacted hydroxyl groups of the porous substrate from the Cholesterol coupling step for further reactions. Then the capping solution was removed and an acidic solution of 3% trichloroacetic acid in dichloromethane (DMT-Removal reagent, Roth, Karlsruhe/Germany, Cat. No. 2257,1) was added for two minutes to remove all remaining DMT groups beside the electrodes. After the exchange of the acidic solution an aqueous solution of ammonia was flushed into the reaction chamber with an incubation time of 60 minutes to deprotect all phosphate protecting groups from phosphate moieties. Finally, some washing steps with acetonitrile and water were performed and a hydrophilic/hydrophobic pattern having hydrophobic properties above the electrodes and hydrophilic properties beside the electrodes was obtained.

After preparation of the two different functionalization patterns the physical properties of the membranes were tested by applying them to an aqueous solution of a 5′-Cy3-(T)₁₅ (SEQ ID NO: 5) oligonucleotide.

After 5 min of incubation time the membrane is washed in a 0.5×SSPE buffer solution and then imaged with a standard digital camera.

The hydrophilic oligonucleotide is moving into the hydrophilic areas of the membrane and tries to avoid the hydrophobic areas. The picture in FIG. 2 shows this behavior were the red oligonucleotide is either on the hydrophilic area above the electrodes (FIG. 2a) or beside the electrodes (FIG. 2b) depending on the functionalization pattern.

### Example 2

### A Porous Substrate with a Hydrophilic/Hydrophobic Pattern in Water

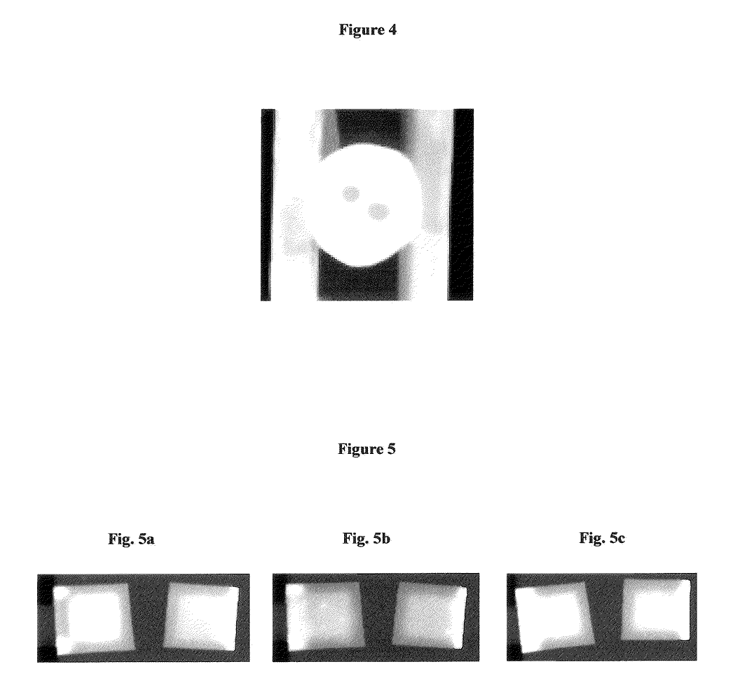

A membrane (PE-Sinter membrane from PolyAn, Berlin/Germany, pore size: 7-16 μm, thickness: 0.6 mm; loading density of hydroxyl groups: 0.8 μmol/cm²) was prepared with a hydrophilic/hydrophobic pattern according to the electrochemical procedure (current—300 μA, deprotection time: 60 sec) mentioned in example 1 with cholesterol moieties above the electrodes. After this preparation, the membrane was placed between two glass slides forming a reaction chamber and water was flushed into this arrangement. The water diffuses into the hydrophilic moieties of the membrane, but not into the hydrophobic areas. FIG. 4 shows the picture of the so treated membrane imaged on a LumiImager instrument (Roche Applied Science, Mannheim, Germany) in the 520 nm channel.

### Example 3

### Change in Fluorescence Intensity Due to Hybridization of Oligonucleotides in a Porous Substrate Using SYBR Green I

Two membranes (PE-Sinter membrane from PolyAn, pore size: 80-130 μm, thickness: 0.6 mm; Loading density of hydroxyl groups: 1.3 μmol/cm²) were prepared with a plastic frame around the edges of the membranes to avoid the leaking of liquid. For this purpose, a solution of polyvinylchloride (PVC) in tetrahydrofurane (THF) was prepared and the edges of the membranes were dipped into this solution. The organic solvent THF evaporates and the PVC remains as a thin film on the membrane. Therefore, liquid dispensed in the middle of such a membrane is captured.

The two membranes were placed on a glass slide and treated with two different end-point PCR solutions from SYBR Green I assays performed with a LightCycler 2.0 instrument (Roche Applied Science, Mannheim, Germany). The two different PCR reactions were performed with SYBR Green I assays from the Universal Probe Library Control Set (Roche Applied Science, Mannheim, Germany, Cat. No. 04 696 417 001; the detailed sequence information are listed in the sequence section) in combination with the LightCycler FastStart DNA MasterPLUS SYBR Green I (Roche Applied Science, Mannheim, Germany, Cat No. 03 515 885) following the standard conditions described in the pack insert.

The first PCR was a positive reaction with primer pairs (SEQ ID NO: 1 and SEQ ID NO: 2) and a synthetic template from the kit mentioned above (Control F from the Universal ProbeLibrary Control Set (Roche Applied Science, Mannheim, Germany), the other PCR a negative control experiment with the same primers but without the template (so called no template control, NTC). The end-point PCR solutions were pipetted in the middle of the both membranes. The membrane on the left of FIG. 5 obtained the positive PCR experiment, the second membrane on the right of FIG. 5 the negative control (NTC). After dispensing the PCR solutions, the membranes were then covered by another glass slide, whereas this reaction chamber was tightened with clips and sealed with adhesive foil.

Afterwards, the fluorescence intensities of the glass slides were recorded with LumiImager instrument (Roche Applied Science, Mannheim, Germany) in the 520 nm channel at two different temperatures, namely at room temperature and at 80° C. Due to the principle that double stranded DNA leads to a fluorescence signal in combination with the intercalating dye SYBR Green I, a large fluorescence signal is present at room temperature for the positive PCR experiment, while only a minor signal can be obtained for the control experiment (see FIG. 5a). At elevated temperature (80° C.) the double stranded DNA is melted and the signal of the SYBR Green I dye disappears, such that both membranes have a minor fluorescence intensity (see FIG. 5b). After a subsequent cooling of the reaction chamber and the formation of double stranded DNA, the fluorescence signal is increasing for positive PCR experiment (see FIG. 5c).

### Example 4

### Change in Fluorescence Intensity by Treatment of Membranes with End-Point PCR Solutions from SYBR Green I Assays

Here, the same experimental setup was used as described in example 3, but with only one membrane (PE-Sinter membrane from PolyAn, pore size: 80-130 μm, thickness: 0.6 mm; Loading density of hydroxyl groups: 1.3 μmol/cm²) that is separated into two compartments by an additional separation line out of polyvinylchloride (PVC) in tetrahydrofurane (THF).

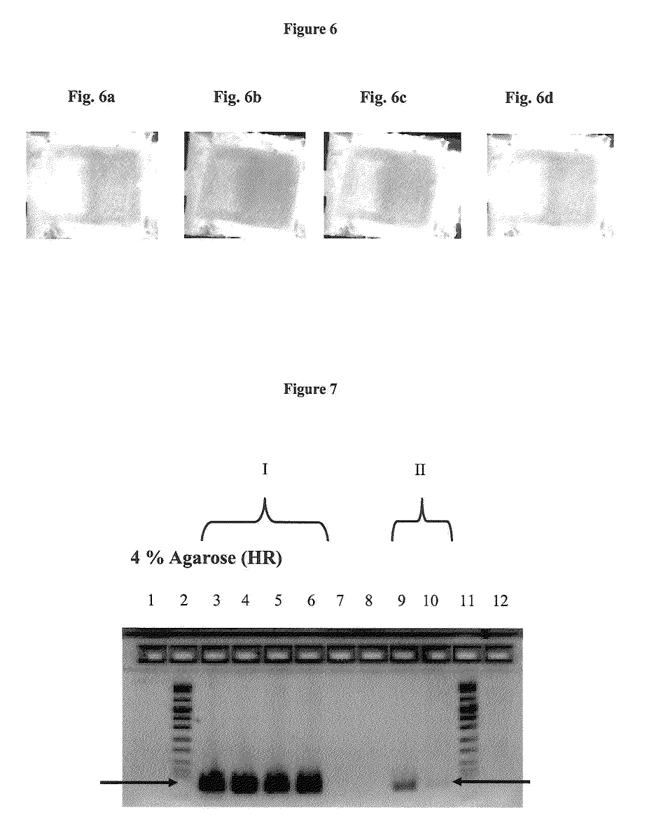

The two solutions from the end-point PCR experiments of Example 3 were pipetted into the two separate compartments of the membrane. Again the membrane was placed in between two glass slides, tightened with clips, sealed with adhesive foil and tempered to room temperature or 80° C. The fluorescence intensities were detected by a LumiImager instrument (Roche Applied Science, Mannheim, Germany) in the 520 nm channel. FIG. 6 shows images of this membrane during a temperature cycle as outlined in example 3 with the corresponding fluorescence behavior (left compartment: positive PCR experiment, right compartment: NTC; a) room temperature, b) 90° C., c) 60° C., d) room temperature).

### Example 5

### PCR Reaction of Beta-2-Microglobulin within a Membrane by Using a Probe-Based Detection Format

A solution of a PCR reaction mixture of beta-2-microglobulin was prepared with a Universal ProbeLibrary assay and an in-vitro RNA transcript (from LightCycler h-β2M Housekeeping Gene Set, Roche Applied Science, Mannheim, Germany, Cat. No. 3 146 081) that was prior reverse transcribed with the Transcriptor first strand cDNA synthesis kit (Roche Applied Science, Mannheim, Germany, Cat. No. 4 379 012) following the standard conditions described in the pack insert. For the final PCR reaction approximately 10⁵ copies of the resulted cDNA were mixed with the primers (SEQ ID NO: 3 and SEQ ID NO: 4), the Universal ProbeLibrary probe (Probe Nr. 42 of the Universal ProbeLibrary) and the PCR master mix (LightCycler TaqMan Master, Roche Applied Science, Cat. No. 04 535 286 001). The final concentrations of all PCR reagents in the PCR mixture were increased in contrast to standard conditions from the pack insert. The final concentrations were as follows: Primers 1000 nM, UPL probe 850 nM, PCR mix 2.1x.

This solution was pipetted onto a membrane (PE-Sinter membrane from PolyAn, pore size: 80-130 μm, thickness: 0.6 mm; Loading density of hydroxyl groups: 1.3 μmol/cm², dimensions 17×14 mm) and then sealed with a plastic foil (commercially available from Tropix Bedford, Mass./USA under the trade name “development folder”, Cat. No. XF030). The membrane was then placed in between two glass slides and the slides were tightened with clips and the PCR reaction was performed under the following conditions: Initial denaturation at 95° C. for 10 min and then 45 amplification cycles each with a denaturation step at 95° C. for 60 sec followed by an amplification step at 65° C. for 90 sec.

After the PCR reaction, the membrane was removed from the plastic foil and the liquid was obtained by centrifugation of the membrane into plastic caps. The obtained solution was pipetted onto a ready-to-use 4% agarose gel (Invitrogen, Carlsbad, Calif./USA, Cat. No. G 501 804) and compared to a PCR reaction as a reference that was done with the same PCR reaction mixture on a LightCycler 2.0 instrument (Roche Applied Science, Mannheim, Germany). On the LightCycler 2.0 instrument the following PCR protocol was used: Initial denaturation at 95° C. for 10 mm and then 45 amplification cycles each with a denaturation step of 95° C. for 10 sec followed by an amplification step of 65° C. for 30 sec and 72° C. for 1 sec with a subsequent final cooling step of 40° C. for 30 sec. FIG. 7 shows the corresponding gel, whereas the bands of two different membrane PCR reactions (indicated by II) gives the same amplicon length as the reference PCR reactions (quadruplicates indicated by I).

Additionally to the gel analysis, the increase of the fluorescence intensities of the membranes were measured due to the cleavage of the fluorophore of the UPL hydrolysis probe during the PCR by a LumiImager instrument (Roche Applied Science, Mannheim, Germany) in the 520 nm channel at the beginning (at cycle 1) and at the end (cycle 45) of the amplification. As expected the signal intensities increased during the PCR reaction from initially 9.8×10⁶ to 1.1×10⁷ for one membrane and from initially 9.6×10⁶ to 1.1×10⁷ for the other membrane in the 520 nm channel.

What is claimed is:

1. A method for nucleic acid amplification comprising
providing a porous substrate having multiple compartments,
adding a nucleic acid containing sample and an amplification mixture to said porous substrate,
exposing said porous substrate to temperature cycles whereby the nucleic acid amplification takes place within the pores of said porous substrate.

2. The method according to claim 1 wherein said porous substrate is provided with at least one attached primer and wherein said amplification mixture comprises enzymes, nucleotides, and buffers.

3. The method according to claim 2 wherein the at least one attached primer is cleaved from the porous substrate prior to performing the temperature cycles.

4. The method according to claim 1 wherein in each of the compartments individual nucleic acid amplifications are performed.

5. The method according to claim 1 wherein the compartments are provided by chemical functionalization of said porous substrate.

6. The method according to claim 1 wherein the compartments are provided by spotting of fluids.

7. The method according to claim 2 wherein a pre-hybridization step is performed prior to exposing the porous substrate to temperature cycles and prior to optional cleaving of the at least one primer from the porous substrate.

8. The method according to claim 1 wherein the porous substrate is sealed in order to avoid cross-talk between the compartments.

9. The method according to claim 1 wherein the porous substrate comprises a material from the group consisting of glass fleece, cellulose, nylon, polyester, polypropylene (PP), polyethylene (PE), poly-ethylenterephthalat (PET), polyacrylnitril (PAT), polyvinylidendifluorid (PVDF), and polystyrene.

10. A porous substrate for nucleic acid amplification comprising multiple compartments to perform a plurality of individual nucleic acid amplifications in parallel,
pores enabling diffusion of nucleic acid molecules and polymerases for nucleic acid amplification within the pores of the porous substrate, and
at least one primer attached to the surface of the porous substrate.

11. The porous substrate according to claim 10 wherein the at least one primer is attached to the porous substrate covalently.

12. The porous substrate according to claim 10 wherein the compartments are defined by chemical barriers, the chemical barriers defined by chemical functionalization of the porous substrate.

13. A multiwell plate for nucleic acid amplification wherein each well of the multiwell plate comprises a porous substrate according to claim 10 such that nucleic acid amplifications take place within the pores of the porous substrates.

14. A kit for nucleic acid amplification comprising
a porous substrate according to claim 10 and
an amplification mixture.

15. A system for nucleic acid amplification comprising
a porous substrate according to claim 10 and
a thermocycler.

16. A system according to claim 15 wherein the thermocycler comprises an illumination means and a detection means.

17. A system according to claim 16 wherein the nucleic acid amplification is a real-time polymerase chain reaction (PCR).
